(19) United States
(12) Patent Application Publication
(10) Pub. No.: US 2006/0084128 A1
(43) Pub. Date: Apr. 20, 2006

(54) Enzyme assay with nanowire sensor

(76) Inventor: Hongye Sun, San Mateo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/231,304

(22) Filed: Sep. 20, 2005

Related U.S. Application Data
(60) Provisional application No. 60/612,315, filed on Sep. 22, 2004.

(51) Int. Cl. C12Q 1/48; C12Q 1/42; C12M 1/34

(52) U.S. Cl. 435/15; 435/21; 435/287.1

(57) ABSTRACT

Systems and methods for enzyme assay using nanowire sensor are disclosed. In some embodiments, a substrate and a group suitable for assaying a target enzyme are identified. The selected substrate is immobilized to a nanowire. The target enzyme introduced to the immobilized substrate modifies the substrate to facilitate addition or removal of the selected group to or from the substrate by formation or breaking of a covalent bond between the group and the substrate. The activity of the target enzyme can be determined by measuring a change in an electrical property of the nanowire due to the addition or removal of the group to or from the immobilized substrate. Kinase and phosphatase are two example reactions that can be assayed by such a method.

### PRIORITY APPLICATIONS

This application claims priority benefit of U.S. Provisional Patent Application No. 60/612,315 filed Sep. 22, 2004, titled “ENZYME ASSAY WITH NANOWIRE SENSOR,” which is incorporated herein by reference in its entirety.

### BACKGROUND

1. Field

The present teachings generally relate to biological assaying techniques, and in particular, to assaying enzymes using nanowire sensors.

2. Description of the Related Art

In many biological assaying applications such as enzyme assays, various parameters can affect the quality and the manner in which an assay is performed. For example, being able to reliably assay a given sample using a relatively small quantity of the sample is usually desired, since the sample may not be available in copious amounts. Also, a high resolution of the assay result is also usually a desirable trait. Furthermore, being able to obtain the assay result in a timely manner is also usually desirable.

While conventional assaying systems and methods exist, there is an ongoing need for improvements in the foregoing and other concerns associated with enzyme assay techniques.

### SUMMARY

The foregoing needs can be addressed by the present teachings, where systems and methods for enzyme assay using nanowire sensor are disclosed. In some embodiments, a substrate and a group suitable for assaying a target enzyme can be identified. The selected substrate can be immobilized to a nanowire. The target enzyme introduced to the immobilized substrate modifies the substrate to facilitate addition or removal of the selected group to or from the substrate by formation or breaking of a covalent bond between the group and the substrate. The activity of the target enzyme can be determined by measuring a change in an electrical property of the nanowire due to the addition or removal of the group to or from the immobilized substrate. Kinase and phosphatase are two example reactions that can be assayed by such a method.

In some embodiments, the present teachings relate to an enzyme assay system that includes a nanowire having a plurality of substrates. The system further includes a plurality of groups that are either charged or have a non-zero electric dipole moment. The system further includes an assay enzyme that chemically modifies a substrate to facilitate formation of a covalent bond between the substrate and a group. Such addition of the group to the substrate results in a change in an electrical property of the nanowire.

In some embodiments, the assay enzyme includes a kinase enzyme. In some embodiments, the substrates modified by the kinase enzyme are reusable by performing a phosphatase reaction.

In some embodiments, the present teachings relate to an enzyme assay system that includes a nanowire having a plurality of substrates with groups covalently bonded thereto. The system further includes an assay enzyme that chemically modifies a substrate to facilitate breaking of a covalent bond between the substrate and a group bonded thereto. Such removal of the group from the substrate results in a change in an electrical property of the nanowire.

In some embodiments, the assay enzyme includes a phosphatase enzyme. In some embodiments, the substrates modified by the phosphatase enzyme are reusable by performing a kinase reaction.

In some embodiments, the present teachings relate to an enzyme assay system that includes a nanowire having a plurality of substrates. The system further includes an assay enzyme that chemically modifies a substrate to facilitate addition or removal of a group to or from the substrate by a formation or breaking of a covalent bond between the substrate and the group. Such a modification to the substrate results in a change in an electrical property of the nanowire.

In some embodiments, the present teachings relate to a method of performing an enzyme assay. The method includes providing an assay enzyme to a plurality of substrates that are part of a nanowire. The assay enzyme chemically modifies a substrate to facilitate addition or removal of a group to or from the substrate by a formation or breaking of a covalent bond between the substrate and the group. The method further includes measuring a change in an electrical property of the nanowire resulting from the addition or removal of the group to or from the substrate. The change in the electrical property of the nanowire is indicative of the number of assay enzymes that chemically modify the substrates.

### BRIEF DESCRIPTION OF THE DRAWINGS

FIG. 1 illustrates a block diagram of an exemplary enzyme assay system having a nanowire;

FIG. 2 illustrates an enlarged block depiction of a portion of the nanowire having immobilized substrates;

FIGS. 3A and B illustrate a chemical modification of the substrate by an enzyme to facilitate formation of a chemical bond between the substrate and a group;

FIGS. 4A and B illustrate different types of groups that can be covalently transferred to the substrate to modify the charge distribution of the substrate and as a result alter the electrical property of the nanowire;

FIGS. 5A and B illustrate a chemical modification of the substrate by an enzyme to facilitate breaking of a chemical bond between the substrate and a group;

FIGS. 6A and B illustrate different types of groups that can be broken from the substrate to modify the charge distribution of the substrate and as a result alter the electrical property of the nanowire;

FIG. 7 illustrates an exemplary measurement of the change in the electrical property of the nanowire as a function of the number of the modified substrates;

FIG. 8 illustrates an exemplary process for preparing the nanowire and performing the enzyme assay using the nanowire;

FIG. 9A illustrates an exemplary process for preparing an array of nanowires for a multiplexed measurement;

FIG. 9B illustrates a block diagram of an exemplary array of nanowires;

FIG. 10A illustrates an exemplary kinase enzyme assay process that is one of a number of possible applications of the process of FIG. 8;

FIG. 10B illustrates an exemplary phosphatase enzyme assay process that is one of a number of possible applications of the process of FIG. 8;

FIGS. 11A and B illustrate an exemplary “real time” enzyme assay of FIGS. 10A and B;

FIG. 12 illustrates an exemplary endpoint enzyme assay of FIGS. 10A and B;

FIG. 13 illustrates an exemplary process where the nanowire can be re-used in kinase/phosphatase enzyme assays;

FIGS. 14A-C illustrate an exemplary assay device having a nanowire based sensor;

FIG. 15 illustrates that a plurality of nanowires having affinities for different enzymes can simultaneously detect the presence of different enzymes; and

FIGS. 16A-D illustrate an exemplary process for fabricating a nanowire sensor.

### DETAILED DESCRIPTION OF SOME EMBODIMENTS

These and other aspects, advantages, and novel features of the present teachings will become apparent upon reading the following detailed description and upon reference to the accompanying drawings. In the drawings, similar elements have similar reference numerals. In this application, the use of the singular includes the plural unless specifically stated otherwise. In this application, the use of “or” means “and/or” unless stated otherwise. Furthermore, the use of the term “comprising,” as well as other forms, such as “comprises” and “comprise,” will be considered inclusive, in that the term “comprising” leaves open the possibility of including additional elements.

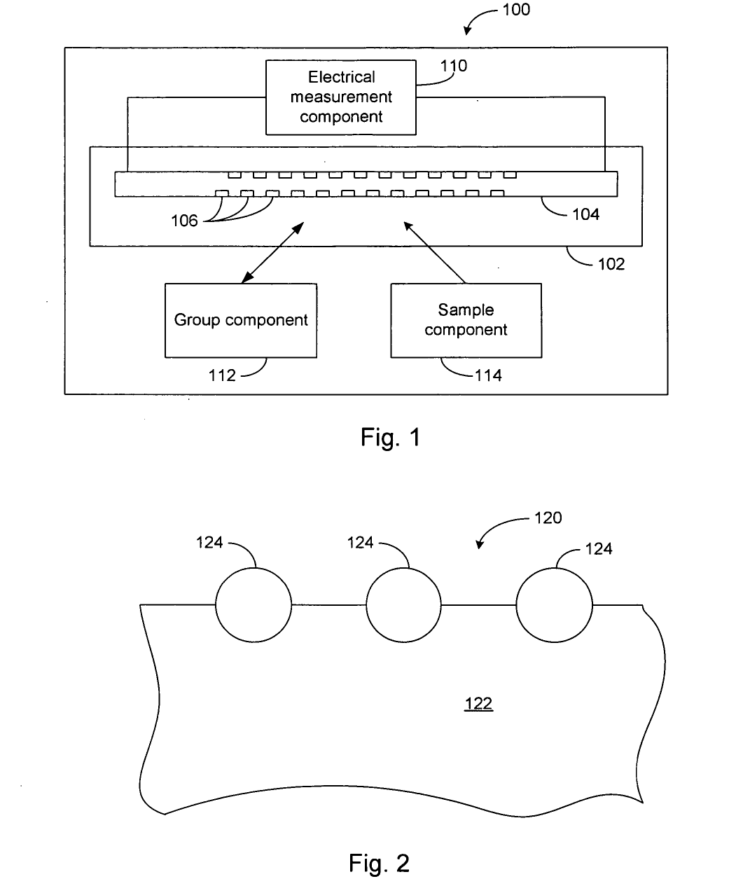

FIG. 1 illustrates an exemplary enzyme assay system 100 comprising one or more nanowire 104. It will be appreciated that the term “nanowire” used herein includes wires, such as silicon nanowires, or tubes, such as carbon nanotubes.

For the purpose of description herein, “nanowire” refers to a nanoscale wire. A “wire” generally comprises one or more materials having an electrical conductivity of a semiconductor or metal. Electrical conductivity refers to the ability of the wire to pass charge. In some embodiments, a nanoscale wire conducts electricity with a resistivity less than or equal to approximately 10⁻³ Ωm, less than or equal to approximately 10⁻⁴ Ωm, or less than or equal to approximately 10⁻⁶ or 10⁻⁷ Ωm.

The “nanoscale” for the purpose of description refers to nanowires having at least one cross-sectional dimension and, in some embodiments, two orthogonal cross-sectional dimensions, less than approximately 1 μm (1000 nanometers). In some embodiments, nanowires have diameters or cross-sectional dimensions of less than or equal to approximately 500 nm, or less than or equal to approximately 200 nm, or less than or equal to approximately 150 nm, or less than or equal to approximately 100 nm, or less than or equal to approximately 70 nm, or less than or equal to approximately 50 nm, or less than or equal to approximately 20 nm, or less than or equal to approximately 10 nm, or less than or equal to approximately 5 nm, or less than or equal to approximately 2 nm, or less than or equal to approximately 1 nm. In some embodiments, a nanowire has at least one cross-sectional dimension, or two orthogonal cross-sectional dimensions, or a diameter, of approximately 0.5 to 200 nm, or 0.5 to 100 nm, or 0.5 to 50 nm, or 0.5 to 25 nm, or 0.5 to 20 nm, or 0.5 to 10 nm, or 1 to 100 nm, or 1 to 50 nm, or 1 to 25 nm, or 1 to 20 nm, or 1 to 10 nm, or 5 to 100 nm, or 5 to 50 nm, or 5 to 25 nm, or 5 to 20 nm, or 5 to 10 nm.

As shown in FIG. 1, some embodiments of the nanowire 104 have a plurality of substrates 106 immobilized thereon. Such substrates 106 can be immobilized onto the nanowire 104 by a number of known techniques.

In some embodiments, the nanowire 104 is coupled to an electrical measurement component 110 that measures an electrical property of the nanowire 104. As is known, one such electrical property comprises electrical conductance G=1/R of the nanowire 104. Other electrical measurements such as current through the nanowire 104, or voltage across the nanowire 104 may be made to detect a change in the electrical property of the nanowire 104.

In some embodiments, the nanowire 104 is disposed within a reaction volume 102 that is adapted to allow addition or removal of group component 112 to and from the substrates 106 in a manner described below. The addition and removal of the group component 112 to and from the substrates 106 can be facilitated by an enzyme sample component 114. In some embodiments, the group component 112 comprises a reaction buffer either having groups therein or adapted to receive groups in a manner described below.

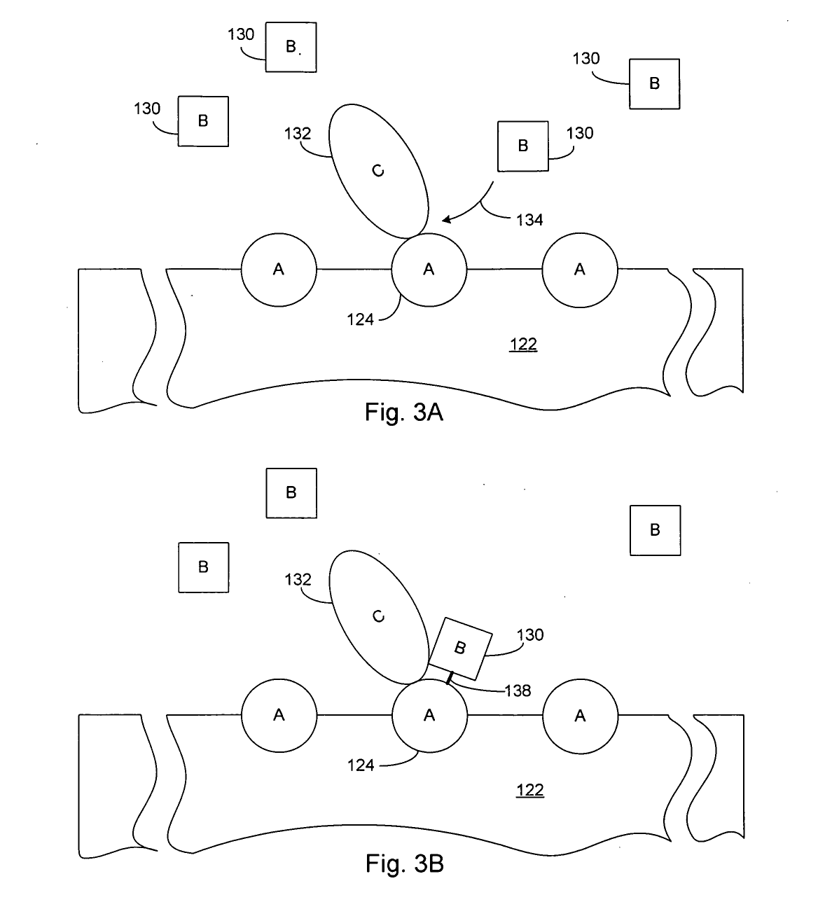

FIG. 2 illustrates an enlarged depiction 120 of a portion of an example nanowire 122. The nanowire 122 is shown to include a plurality of substrates 124 immobilized with respect to the nanowire 122. As previously described, the substrates 124 may be immobilized with respect to the nanowire 122 by any number of known techniques.

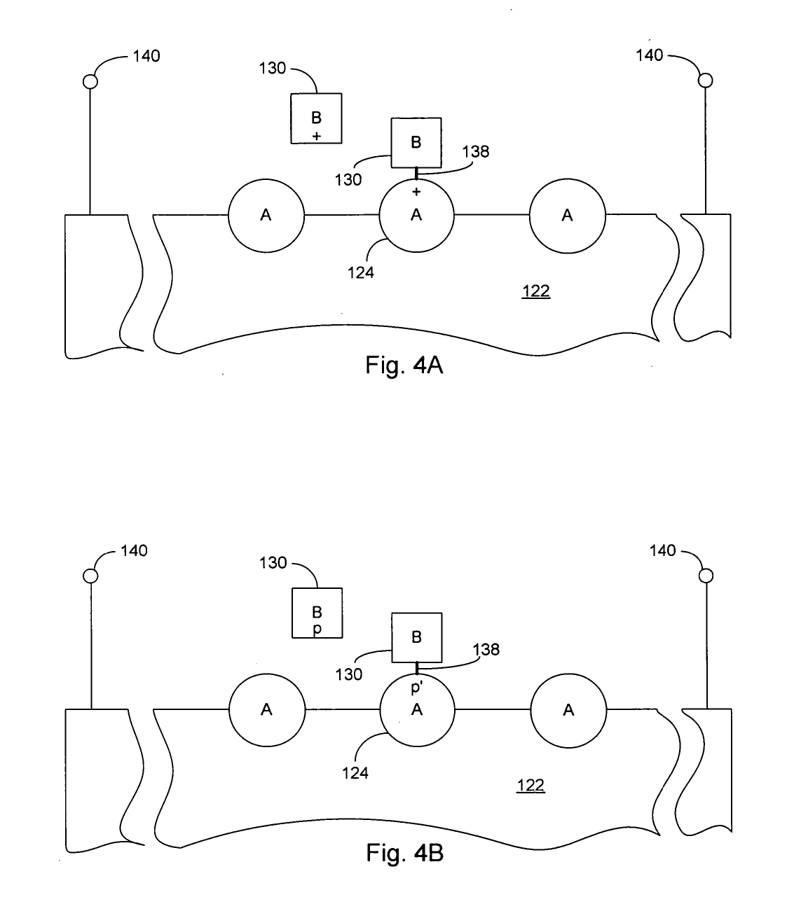

FIGS. 3A and B now illustrate how an exemplary group “B” 130 can be added to the substrate “A” 124. In some embodiments, the nanowire 122 having immobilized substrates 124 is disposed in an environment having a plurality of groups 130. A selected target enzyme “C” 132 can interact with the substrate 124 and group 130 and chemically modify the substrate 124 to facilitate (as indicated by an arrow 134) formation of a covalent bond 138 between the substrate 124 and the partial or whole group 130. Thus, a modified substrate is indicative of activity of the target enzyme, and detection of the modified substrate allows quantification of the target enzyme activity. In some embodiments, the detection of the modified substrate is achieved by detecting a change in an electrical property of the nanowire 122.

The group 130 may comprise charged groups, or groups having electrical dipole moment. The group that is covalently transferred to the immobilized substrates on the nanowire may be part or the whole group 130. The charged group may include, but not limited to, phosphate, sulfate, DNA, RNA, amino acids and the like. The groups having electrical dipole moment may include, but not limited to, sugar, amino acids, and the like. In some embodiments, the substrates 124 comprise peptide, protein, DNA/RNA, and other small molecules.

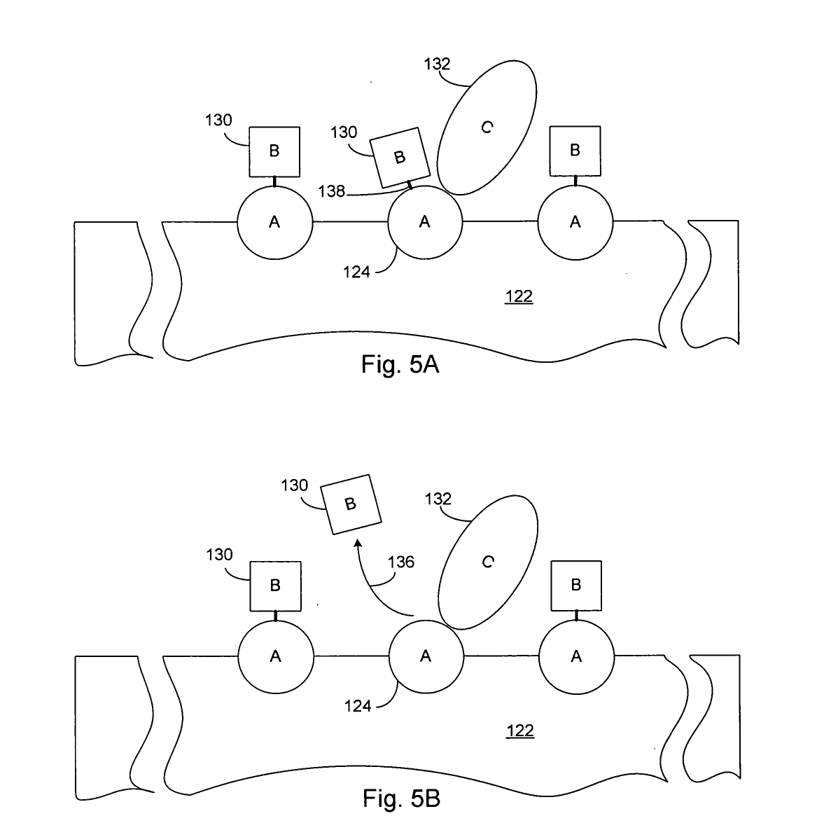

FIGS. 4A and B illustrate how the charged group or the dipole group of the group 130 can modify the charge distribution of the substrate 124. In FIG. 4A, the group 130 comprises the charge group, as indicated by a “+” sign. It will be understood that usage of the “+” sign is to indicate that the group 130 is charged, and is in no way intended to limit the charge to +1. The charge of the group 130 may be plus or minus any non-zero integer associated with the charged group. The addition of the group 130 to the substrate 124 results in a change in the charge distribution of the substrate 124, which in turn can result in a change in the electrical property associated with the nanowire 122. As an example, the conductance of the nanowire 122 between source and drain points (both indicated as 140) may change in a measurable manner.

In FIG. 4B, the group 130 comprises the dipole group, as indicated by a “p” symbol. The addition of the group 130 to the substrate 124 can induce a dipole moment “p′” in the substrate 124, thereby changing the charge distribution of the substrate 124. The change in the substrate's charge distribution in turn results in a change in the electrical property associated with the nanowire 122 (again, measured between the source/drain points 140).

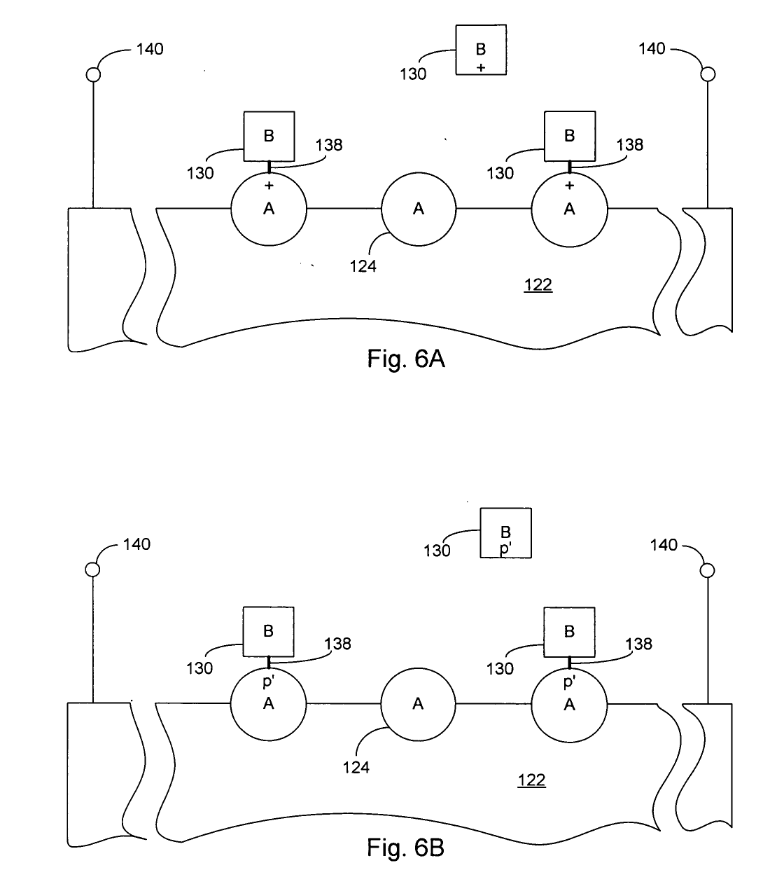

FIGS. 5A and B now illustrate how an exemplary group “B” 130 can be removed from the substrate “A” 124. In some embodiments, the nanowire 122 is prepared such that the substrates 124 have groups 130 bonded thereto. In some embodiments, such a nanowire 122 is disposed in an environment adapted to receive the groups 130 that can be liberated from the substrates 124. A selected target enzyme “C” 132 can interact with the substrate 124 and chemically modify the substrate 124 to facilitate (as indicated by an arrow 136) breaking of a covalent bond 138 between the substrate 124 and the group 130. Thus, a groupless substrate is indicative of the activity of a target enzyme, and detection of such modified substrate allows quantification of the target enzyme activity. In some embodiments, the detection of the modified substrate is achieved by detecting a change in an electrical property of the nanowire 122.

The group 130 may comprise charged groups, or groups having electrical dipole moment. The charged group may include, but not limited to, phosphate, sulfate, DNA, RNA, and the like. The groups having electrical dipole moment may include, but not limited to, sugar, amino acids, and the like. In some embodiments, the substrates 124 comprise peptide, protein, DNA/RNA, and other small molecules.

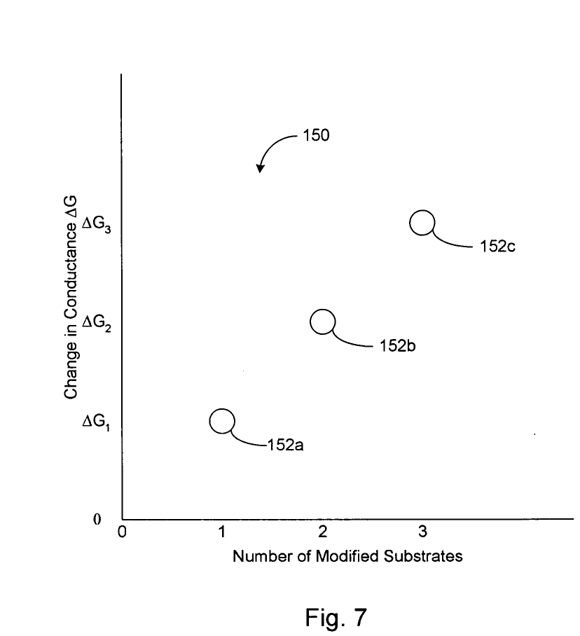

FIGS. 6A and B illustrate how the removal of the charged group or the dipole group of the group 130 can modify the charge distribution of the substrate 124. In FIG. 6A, a liberated group 130 comprises the charge group, as indicated by a “+” sign. It will be understood that usage of the “+” sign is to indicate that the group 130 is charged, and is in no way intended to limit the charge to +1. The charge of the group 130 may be plus or minus any non-zero integer associated with the charged group. The removal of the group 130 from the substrate 124 results in a change in the charge distribution of the substrate 124, which in turn can result in a change in the electrical property associated with the nanowire 122. As an example, the conductance of the nanowire 122 between source and drain points (both indicated as 140) may change in a measurable manner.

In FIG. 6B, the liberated group 130 comprises the dipole group, as indicated by a “p” symbol. The removal of the group 130 from the substrate 124 removes an induced dipole moment “p′” in the substrate 124, thereby changing the charge distribution of the substrate 124. The change in the substrate's charge distribution in turn results in a change in the electrical property associated with the nanowire 122 (again, measured between the source/drain points 140).

As described above in reference to FIGS. 3-6, the modification of a substrate 124 by addition or removal of a group 130 results in a change in the nanowire's electrical conductance. In some embodiments, the measurement of such a change in conductance may be performed with sufficient accuracy to yield a single molecule resolution.

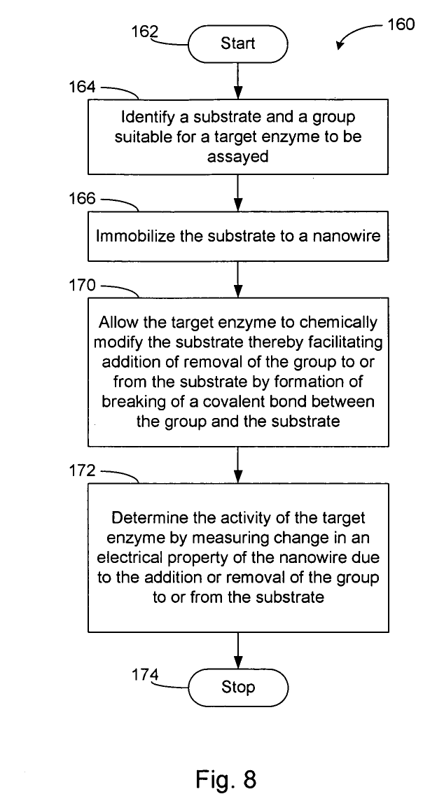

FIG. 7 illustrates an exemplary relationship 150 between a conductance change ΔG as a function of the number of modified substrates. The exemplary relationship 150 is representative of some embodiments of an assay system capable of a single molecule resolution, such that one target enzyme molecule/substrate combination results in a conductance change of ΔG₁ (exemplary data point 152a).

The desired resolution of a nanowire based assay application, as well as the types of substrates and groups used, depend on the assay to be performed. As an example, many useful enzyme reactions involving addition or removal of groups fall under kinase and phosphatase reactions. In such assays, the group may comprise a phosphate group, and the substrate that is modifiable by a target enzyme to be receptive to the phosphate may be selected accordingly. Various advantages of using the nanowire assay system for such reactions are described below in greater detail.

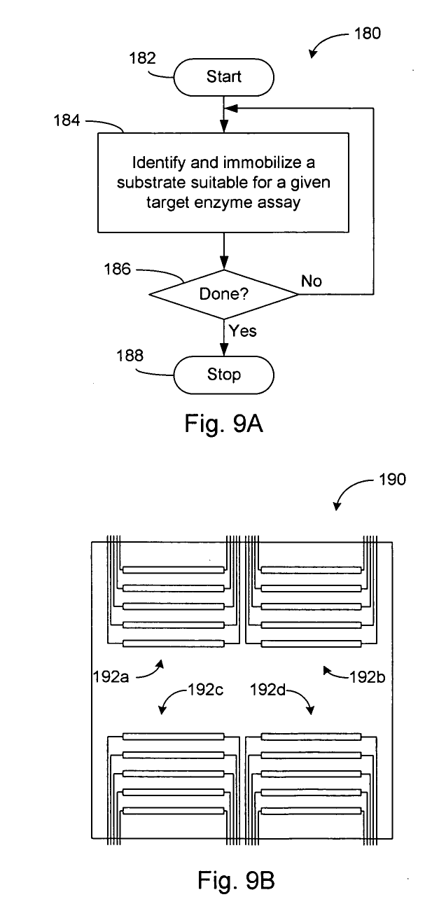

FIG. 8 now illustrates an exemplary process 160 of performing an enzyme assay using the nanowire. The process 160 begins in a start state 162, and in step 164 that follows, one identifies a substrate and a group suitable for a target enzyme to be assayed. In step 166 that follows, the process 160 immobilizes the selected substrate to the nanowire. In step 170 that follows, the process 160 allows the target enzyme chemically modify the substrate thereby facilitating addition or removal of the group to or from the substrate by formation or breaking of a covalent bond between the group and the substrate. In step 172 that follows, the process 160 determines the activity of the target enzyme by measuring a change in an electrical property of the nanowire due to adding or removing of the group to or from the substrate. The process 160 ends in a stop state 174.

It will be understood that the exemplary process 160 does not have to occur in a continuous manner. For example, the “preparation” of the nanowire (steps 164 and 166) may be performed as a separate operation from the “reaction/measurement” phase (steps 170 and 172) of the assay.

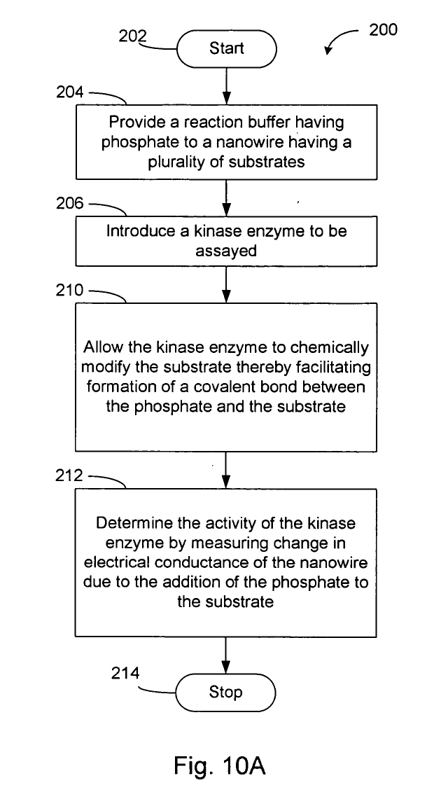

FIGS. 9A and B now illustrate that the nanowires can be formed in an array format to allow multiplexed reaction measurements. FIG. 9A illustrates an exemplary process 180 for preparing an array of nanowires having appropriate immobilized substrates. The process begins in a start state 182, and in step 184, the process 180 identifies and immobilizes a substrate suitable for a given target enzyme assay. In a decision step 186 that follows, the process 180 determines whether the array preparation is completed. If the answer is “yes,” the process 180 ends in a stop state 188. If the answer is “no,” the process 180 proceeds to preparation of another nanowire as described in step 184.

FIG. 9B illustrates an exemplary array 190 that may be formed by the exemplary process 180 of FIG. 9A. The exemplary array 190 depicts four exemplary groups (192a, b, c, d) of nanowires. Each group may be configured to be sensitive to a particular type of target enzyme and group, and may comprise a plurality of nanowires. It will be appreciated that the exemplary array of FIG. 9B is just that—exemplary for the purpose of description. Any other array configurations may be used.

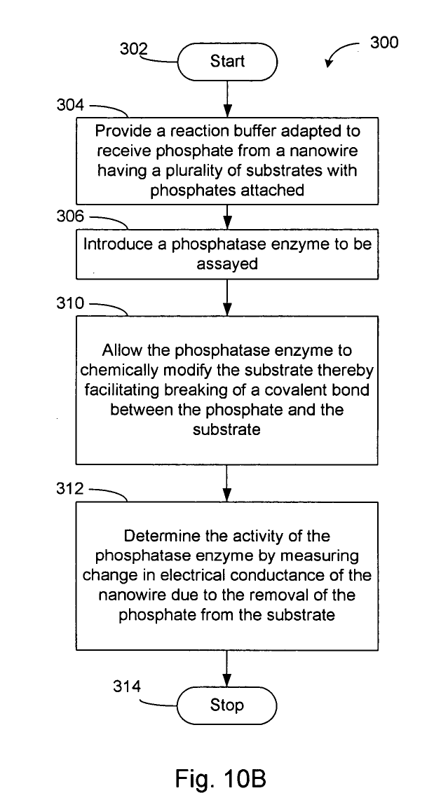

FIGS. 10-13 now illustrate various aspects of the application of the nanowire based assay system to kinase and phosphatase reactions. The exemplary kinase/phosphatase reaction applications are described in terms of a nanowire. It should be understood, however, that similar assays are also applicable with the array of nanowires.

FIG. 10A illustrates an exemplary process 200 that is a kinase reaction application of the process 160 described above in reference to FIG. 8. The kinase assay process 200 begins in a start state 202, and in step 204 that follows, the process 200 provides a reaction buffer having a plurality of ATP (Adenosine 5′-triphosphate) groups to a nanowire having a plurality of substrates. In step 206 that follows, the process 200 introduces a kinase enzyme to be assayed to the nanowire. In step 210 that follows, the process 200 allows the kinase enzyme to chemically modify the substrate thereby facilitating formation of a covalent bond between the γ phosphate and the substrate. In step 212 that follows, the process 200 determines the activity of the kinase enzyme by measuring the change in the electrical conductance of the nanowire due to the addition of the phosphate to the substrate. The process ends in a stop state 214.

FIG. 10B illustrates an exemplary process 300 that is a phosphatase reaction application of the process 160 described above in reference to FIG. 8. The phosphatase assay process 300 begins in a start state 302, and in step 304 that follows, the process 300 provides a reaction buffer adapted to receive phosphate from a nanowire having a plurality of substrates with phosphates attached. In step 306 that follows, the process 300 introduces a phosphatase enzyme to be assayed to the nanowire. In step 310 that follows, the process 300 allows the phosphatase enzyme to chemically modify the substrate thereby facilitating breaking of a covalent bond between the phosphate and the substrate. In step 312 that follows, the process 300 determines the activity of the phosphatase enzyme by measuring the change in the electrical conductance of the nanowire due to the removal of the phosphate from the substrate. The process ends in a stop state 314.

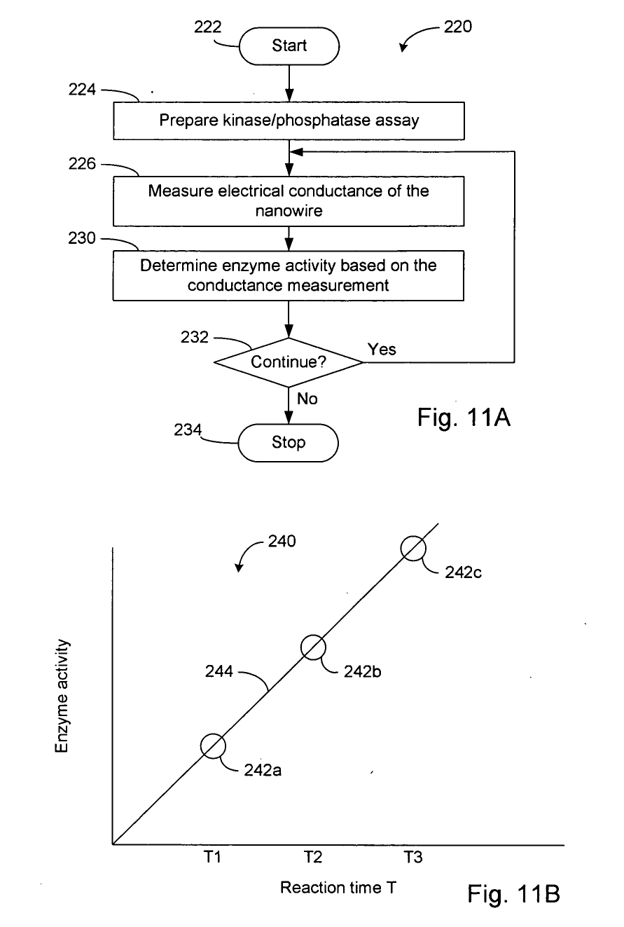

The exemplary kinase/phosphatase assay processes 200 and 300 of FIGS. 10A and B may be performed in substantially “real time” to study the time progression of the enzyme activity, or as an end point enzyme activity measurement. FIG. 11A illustrates an exemplary process 220 that performs a substantially real time monitoring of the kinase/phosphatase activity. The process 220 begins in a start state 222, and in step 224 that follows, the process 220 prepares the kinase/phosphatase assay. Such preparation may include preparing the nanowire and providing a suitable reaction buffer to the nanowire, and introducing a kinase/phosphatase enzyme to the nanowire. The process 220 in step 226, measures the electrical conductance of the nanowire at a predetermined time. In step 230 that follows, the process 220 determines the kinase/phosphatase activity based on the conductance measurement. Such substantially “real time” kinase/phosphatase activity may be recorded and/or displayed. In a decision step 232 that follows, the process 220 determines whether the kinase/phosphatase activity monitoring should continue. If the answer is “yes,” the process 220 loops back to the conductance measurement step 226 for another measurement at a predetermined time. If the answer is “no,” the process 220 ends in a stop state 234.

FIG. 11B illustrates an exemplary relationship 240 between the enzyme activity and the reaction time T, as obtained by the process 220 of FIG. 11A. Exemplary enzyme activity data points 242a, b, and c corresponding to reaction times T1, T2, and T3, may yield useful information about the progression of the enzyme activity (for example, a linear relationship 244).

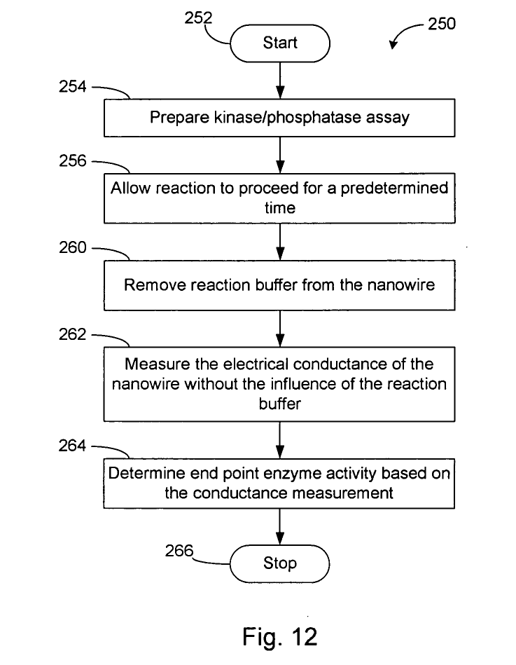

FIG. 12 now illustrates an exemplary process 250 that performs an end point kinase/phosphatase activity measurement. The process 250 begins in a start state 252, and in step 254 that follows, the process 250 prepares the kinase/phosphatase assay. In step 256 that follows, the process 250 allows the reaction to proceed for a predetermined time to reach a substantially steady state. In step 260 that follows, the process 250 removes a reaction buffer from the nanowire. In step 262 that follows, the process 250 measures the electrical conductance of the nanowire without the influence of the reaction buffer. In step 264 that follows, the process 250 determines the end point enzyme activity based on the conductance measurement. The process 250 ends in a stop state 266.

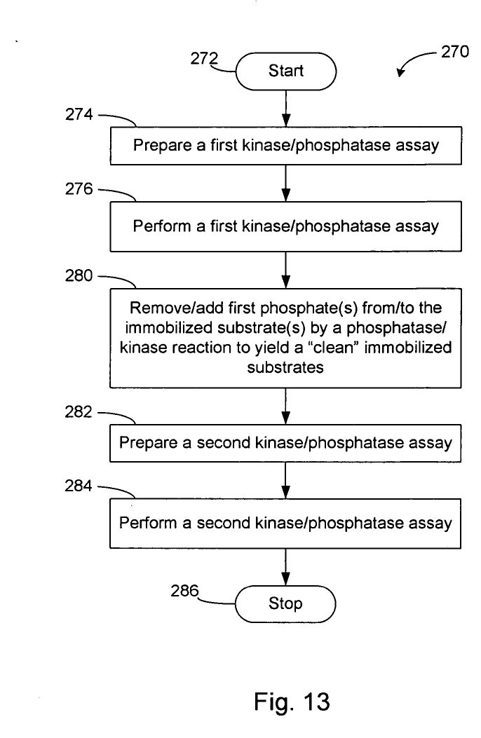

FIG. 13 now illustrates an exemplary process 270 where the nanowire used for a kinase/phosphatase assay can be reused for subsequent assays. The process 270 begins in a start state 272, and in step 274 that follows, the process 270 prepares a first kinase/phosphatase assay. In step 276 that follows, the process 270 performs the first kinase/phosphatase assay. In step 280 that follows, the process 270 removes/adds the first phosphate(s) from/to the immobilized substrate(s) by a phosphatase/kinase reaction to yield a “clean” immobilized substrates. In steps 282 and 284 that follow, the process 270 prepares and performs a second kinase/phosphatase assay. The process 270 can either repeat step 280 for a third (and so on) assay, or end in a stop state 286.

An example of a kinase assay using a nanowire sensor may include protein kinase A (PKA). Protein kinase A peptide substrate with amino acid sequence NH₂-LRRASLG-COOH can be immobilized on the nanowire by covalent attachment through the N-terminal amine —NH₂ or C-terminal —COOH or non-covalent by attaching a biotin group on either end of the peptide substrate such that strepatividin is attached onto the nanowire. A reaction buffer may comprise, for example, 20 mM Tris-HCl (pH 7.5), 10 mM Mg²⁺, 0.1 nM PKA, and 2 uM ATP. Incubating the reaction buffer with the nanowires coated with substrate results in the γ-phosphate group covalently attaching to the hydroxyl group of the serine residue in the peptide substrate, which changes the charge status of the substrate. The buffer composition and the concentrations of ATP, enzyme and other constituents can be optimized according to standard biochemical practices.

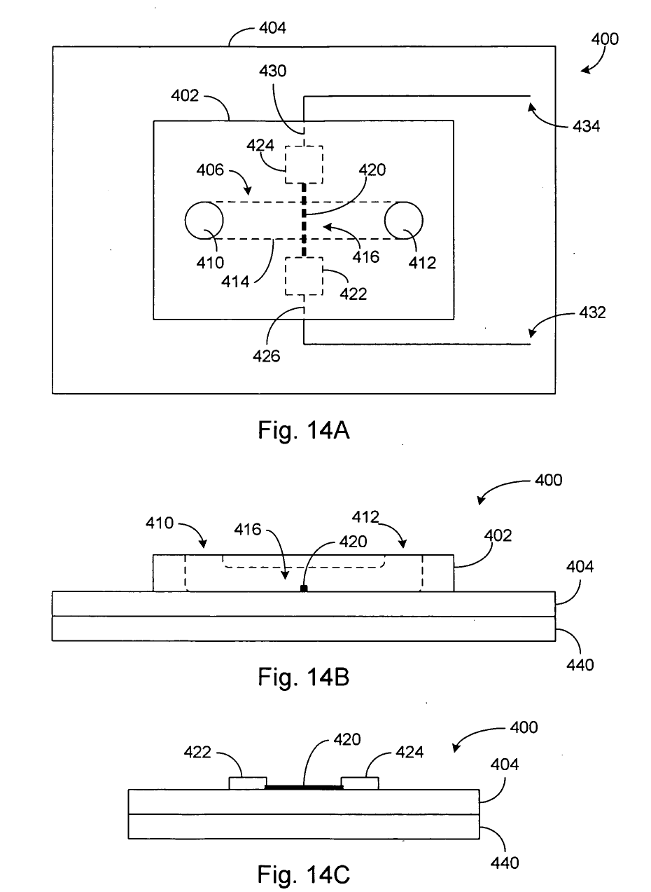

FIGS. 14-16 now illustrate an exemplary nanowire based sensor that can be used to perform the above described assays. FIG. 14A illustrates a block diagram of an exemplary sensor 400 having a nanowire 420 overlapping a reaction volume 416. In some embodiments the reaction volume 416 comprises a microchannel 414 defined by a reaction block 402. The microchannel 414 includes an input area 410 and an output area 412 that allow input and output to introduce the various reaction components to the reaction volume.

As further shown in FIG. 14A, the nanowire 420 can be connected to a source 422 and a drain 424 that are respectively connected to conducting elements 426 and 430. In some embodiments, the conducting elements 426 and 430 may terminate at respective contact points 432 and 434 to allow electrical connections with other components for the operation and/or measurement of the nanowire 420.

In some embodiments, the reaction block 402 can be formed on a substrate 404. The reaction block 402 can be formed by fabricating a master by using photolithography and casting a polydimethylsiloxane (PDMS) mold. In the exemplary mold (block 402) shown in FIG. 14B, the mold defines the exemplary reaction volume 416 that overlaps a portion of the nanowire 420. The reaction components can enter the reaction volume 416 through the input area 410, and leave through the output area 412.

FIG. 14C illustrates an exemplary layout of the nanowire 420 on the substrate 404 (reaction block 402 not shown), terminated at the source 422 and the drain 424. The nanowire 420 can be formed in an exemplary manner described below.

As further illustrated in FIGS. 14B and C, some embodiments of the sensor may include a back gate layer 440 that provides a back gate voltage to the nanowire 420 thereby controlling the sensitivity of the nanowire 420. In some embodiments, the substrate 404 may comprise a layer of silicon oxide, and the back gate layer 440 may comprise a silicon layer.

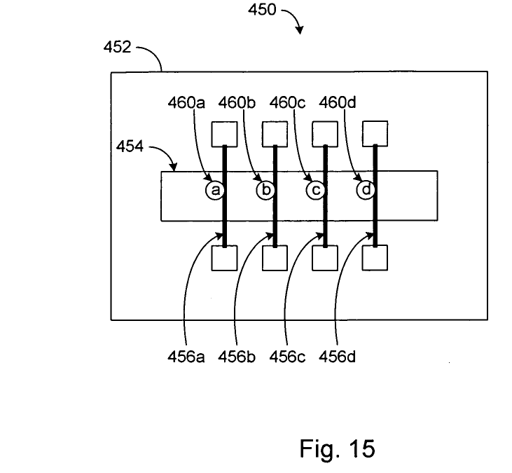

FIG. 15 illustrates an exemplary sensor 450 having a plurality of nanowires 456 disposed on a substrate 452. The plurality of nanowires 456 are shown to be connected to their respective sources and drains. The plurality of nanowires 456 are also shown to overlap with a common reaction volume 454. In some embodiments, each of the plurality of nanowires 456 may be adapted to have an affinity for a selected enzyme. Thus, an enzyme “a” at 460a may have an affinity to the nanowire 456a, and so on. By having such an array of nanowires 456 in contact with the common reaction volume 454, one can perform an assay on multiple enzymes simultaneously. A plurality of nanowires of various configurations can be arranged in any number of ways with respect to one or more reaction volumes to achieve a desired assay configuration.

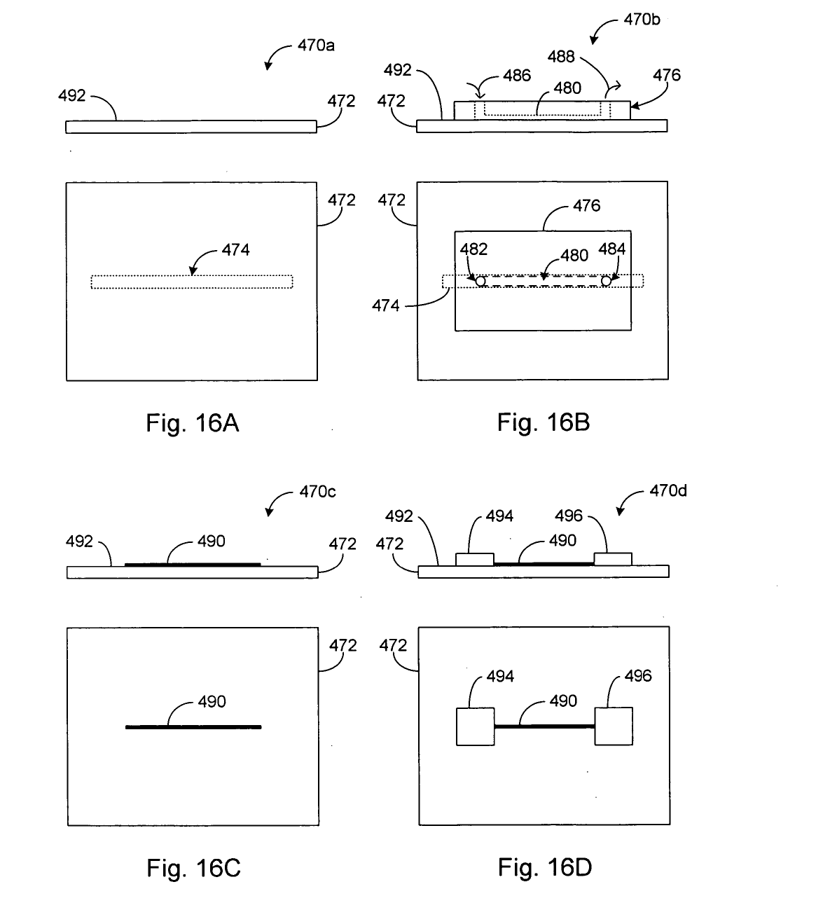

FIG. 16 now illustrates one way of fabricating the exemplary nanowire on the substrate described above in reference to FIGS. 14 and 15. In one stage 470a of the fabrication, a selected portion 474 of a first surface 492 of a substrate layer 472 can be chemically modified to promote adhesion of nanowire fragments. In a subsequent stage 470b, a solution having a suspension of nanowire fragments can be passed over the chemically modified portion 474. For example, one way of introducing the solution is to form a channel 480 adjacent the modified portion. The channel 480 can be formed using a PDMS mold referred to above, and the solution can be introduced to and removed from the channel 480 via an input (arrow 486) and an output (arrow 488).

In certain embodiments, the introduction of nanowire fragment suspension to the modified portion 474 with an application of a bias voltage to the substrate 472 results in the nanowire fragments adhering to the modified portion 474, thereby forming a nanowire structure 490 (as shown in a fabrication stage 470c in FIG. 16C). As shown in a fabrication stage 470c of FIG. 16D, a source 494 and a drain 496 can be formed at the ends of the nanowire structure 490 to be used as exemplary sensors described above in reference to FIGS. 14 and 15.

In addition to the discussion herein, further guidance concerning nanowires and the fabrication thereof which may be modified to practice the teachings herein can be found in a U.S. patent application Ser. No. 10/020,004 entitled “Nanosensors” filed Dec. 11, 2001 and Ser. No. 10/196,337 entitled “Nanoscale Wires and Related Devices” filed Jul. 16, 2002, and Yi et al., Science 293:1289-1292 (2001), which are hereby incorporated by reference.

Although the above-disclosed embodiments of the present invention have shown, described, and pointed out the fundamental novel features of the invention as applied to the above-disclosed embodiments, it should be understood that various omissions, substitutions, and changes in the form of the detail of the devices, systems, and/or methods illustrated may be made by those skilled in the art without departing from the scope of the present invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. An enzyme assay system comprising:
a nanowire having a plurality of substrates;
a plurality of groups that are either charged or have a non-zero electric dipole moment; and
an assay enzyme that chemically modifies a substrate to facilitate formation of a covalent bond between the substrate and a group wherein such addition of the group to the substrate results in a change in an electrical property of the nanowire.

2. The system of claim 1, wherein the assay enzyme comprises a kinase enzyme.

3. The system of claim 2, wherein the substrates modified by the kinase enzyme are reusable by performing a phosphatase reaction.

4. An enzyme assay system comprising:
a nanowire having a plurality of substrates with groups covalently bonded thereto; and
an assay enzyme that chemically modifies a substrate to facilitate breaking of a covalent bond between the substrate and a group bonded thereto wherein such removal of the group from the substrate results in a change in an electrical property of the nanowire.

5. The system of claim 4, wherein the assay enzyme comprises a phosphatase enzyme.

6. The system of claim 5, wherein the substrates modified by the phosphatase enzyme are reusable by performing a kinase reaction.

7. An enzyme assay system comprising:
a nanowire having a plurality of substrates; and
an assay enzyme that chemically modifies a substrate to facilitate addition or removal of a group to or from the substrate by a formation or breaking of a covalent bond between the substrate and the group wherein such a modification to the substrate results in a change in an electrical property of the nanowire.

8. A method of performing an enzyme assay, the method comprising:
providing an assay enzyme to a plurality of substrates that are part of a nanowire wherein the assay enzyme chemically modifies a substrate to facilitate addition or removal of a group to or from the substrate by a formation or breaking of a covalent bond between the substrate and the group; and
measuring a change in an electrical property of the nanowire resulting from the addition or removal of the group to or from the substrate wherein the change in the electrical property of the nanowire is indicative of the number of assay enzymes that chemically modify the substrates.
